(19) United States
(12) Patent Application Publication
(10) Pub. No.: US 2011/0313326 A1
(43) Pub. Date: Dec. 22, 2011

(54) Apparatus and Method for Testing Muscular Power Capacity

(76) Inventor: Karete Hoiberg Johansen, Grimstad (NO)

(21) Appl. No.: 13/129,089

(22) PCT Filed: Nov. 9, 2009
(86) PCT No.: PCT/NO2009/000386

§ 371 Date: Jun. 14, 2011

(30) Foreign Application Priority Data
Nov. 14, 2008 (NO) 20084807

(51) Int. Cl. A61B 5/22; A61B 5/11

(52) U.S. Cl. 600/595

(57) ABSTRACT

Apparatus for use when assessing muscular strength and body posture in a body core, wherein at least a portion of the apparatus is arranged to be attached to a body; the apparatus is arranged to be substantially fixedly positioned relative to two anatomical landmarks; the apparatus is provided with at least one of a sensor and a signal transmitter for measuring an angle between a line through the two anatomical landmarks and a reference line which is independent of the body; and the apparatus is connected to an angle indicator.

The present invention relates to an apparatus which helps to assess muscular strength and provide information on body posture. More particularly, the invention relates to an apparatus arranged to be attached to the body, the apparatus being provided with at least one sensor for measuring an angle between a line through two anatomical landmarks and a reference line. Further, the invention relates to a method of providing a measured value when measuring the muscular power of a muscle region in a person, and use of the apparatus.

By the core of the body, the body core or the core musculature of the body are meant the central muscles in the abdominal and back regions. The core musculature consists of superficial and deep spine musculature, abdominal musculature, pelvic floor musculature and musculature surrounding the hips. Thus, the core is the key musculature of the body for all body movement and stabilization to a greater or lesser degree. Knowledge about the core and the function of the core with respect to the stability of the body is central in therapeutic treatment.

By a kinetic chain are meant joints and body segments which are connected in series, connected to the same power unit. That is to say a chain which can resist/create rotation in the core.

By a body-weight exercise is/are meant an exercise or exercises using the body's own weight as training resistance.

By the neutral zone of the vertebral column is meant that the vertebrae are held in position relative to each other without passive structures like ligaments, cartilage and cartilage discs contributing significantly to their stabilization.

By the term spinal stability is meant the connection between three subsystems: the passive vertebral column, the active back musculature and the nervous control.

By the local stabilizer system are meant deep musculature lying close to the vertebral column and deep abdominal musculature forming the inner pelvic wall. By the global stabilizer system are meant large, outer, superficial muscles around the abdominal and back regions.

By core strength is meant the capacity to voluntarily create rotation in the core, so that the position and movement of the truncus above the pelvis and lower extremities are maximized with a view to optimal power development capacity, power transmission capacity and capacity to control the power and movement of the peripheral body segments in the activities of the integrated kinetic chain.

By core stability is meant the capacity to resist involuntary rotation in the core, so that control of the position and movement of the truncus above the pelvis and lower extremities is maintained to enable optimal development of, trans-mission of and control of the power and movement of the peripheral body segments in the activities of the integrated kinetic chain.

By core power is meant the sum of core strength and core stability.

By a “weak link” is meant a deviation in the biomechanical chain. This may be due to reduced neuromuscular control or reduced muscular power or conscious avoidance of something that seems to create fear, so-called “fear avoidance”. If the body has weakenings, “weak links”, in the kinetic chain, that may lead to weakened performances and pathology.

By an inclinometer is meant a sensor in the form of an angle gauge. Alternative names for an inclinometer include tilt meter, clinometer and gradiometer.

By the sagittal plane is meant a plane which divides the body into a right half and a left half. By the frontal plane is meant a plane which divides the body into a front half and a rear half. The frontal plane is also referred to as the coronal plane. By the transverse plane is meant a plane which divides the body into an upper half and a lower half. In anatomical movements, these planes will describe around what axis or in what planar direction the movement takes place. Movement of the human body in these three planes and around the relevant three axes at different speeds and different torques and forces makes great demands on strength, endurance and coordination of the core musculature of the body.

The functional capacity and the power of the musculature in the core region can be tested. There are a number of test methods for measuring the power capacity of the core. These methods are well discussed in the specialist literature. It is common knowledge that it is not easy to quantify the capacity of the human body to create stability and strength in the core region in an objective, reproducible, accurate, reliable and valid way. Further, there is often uncertainty in connection with how the results achieved are to be interpreted because of the lack of default values for a population or at least reference values for the test person. Further, it has been known for a long time that it is difficult to find unambiguous and measurable connections between repeated test results in connection with the power capacity of the core region by the use of well-established and standardized clinical test methodology, including “weak-link” tests. Examples of “weak-link” tests are the “supine abduction test” and the one-leg squat test. The weaknesses existing in the known tests and their lack of connections with respect to measuring results seem to be caused by the fact that different dimensions of the core power of the body are measured.

Norm material for what healthy persons should be able to achieve in, for example, the “supine abduction test” before the pelvis rotates around a longitudinal axis (an axis extending from the head to the toes) is largely absent. The need for defining default values is thereby large. In a clinical connection, a subjective, visually based method has been used until now, which, in reality, is not very suitable. The need for an objective measuring method which is also a practical one has been a long-felt need for physiotherapists who use sling-based therapy. The need is also large in view of research purposes. The test has been difficult to use reproducibly across individuals and across test supervisors. It has also been difficult to demonstrate improvement or deterioration in a condition in one individual by retesting.

The invention has for its object to remedy or reduce at least one of the drawbacks of the prior art.

The object is achieved through features which are specified in the description below and in the claims that follow.

The invention takes for its starting point that, on the body, it is possible to select points that are substantially fixed relative to each other during different body movements. They are termed anatomical landmarks herein. Examples of such landmarks, which are rigidly connected and easy to identify without possessing any special anatomy knowledge, are points like the “spinae iliacae anteriores superiores” (SIAS; the front, upper iliac crest), ribs in pairs, points on the skull, for example ear-ear, and points on the extremities, for example the medial and lateral ankle knuckles on the same foot. An example of anatomical landmarks which are not rigidly connected is the navel and the 5^{th} lumbar vertebra of the spine. In accordance with the object of the invention, it is appropriate to choose anatomical landmarks on the truncus.

Core power is, as previously described, both the creating and the resisting of rotation in the core, so that control of the position and movement of the truncus above the pelvis and lower extremities is maintained.

The invention further utilizes the capacity of the core to create or resist rotation. The two anatomical landmarks selected define a line which has a spatial direction. This line will form an angle with a defined plane or with a defined line lying in that plane. The local gravity defines horizontal planes and vertical planes. Measuring the core power will then be the measuring of whether the core musculature is able to hold the line between the two anatomical landmarks at a fixed angle relative to a selected plane or a line in that plane, especially when the test person is subjected to a lever. Alternatively, the capacity to create rotation can be measured by the size of the angle through which the core is able to move the line between the two selected anatomical landmarks relative to a selected plane or a selected line in that plane.

Measuring an angle between a line and a plane or a line in the plane can be done with, for example, an inclinometer, a vial of a level or with an accelerometer. When an electronic accelerometer is used, the signal must be processed so that integration of the data provides an angle reading. Two levels may be combined into measuring angles in two planes simultaneously, and three levels may be combined into measuring in three planes simultaneously by being set mutually perpendicular. Correspondingly, two and three accelerometers can measure in two and three planes, respectively. Levels, accelerometers and other angle-measuring devices may also be combined.

Accelerometers will also provide additional information about unsteadiness in the movement. This may be used to provide more information about, inter alia, the neuromuscular control as the exercise is performed. Separate accelerometers may also be placed elsewhere on the body if appropriate.

In a first aspect, the invention comprises an apparatus for use when assessing muscular strength and body posture in a body core, wherein:
at least a portion of the apparatus is arranged to be attached to a body;
the apparatus is arranged to be substantially fixedly positioned relative to two anatomical landmarks;
the apparatus is provided with at least one of a sensor and a signal transmitter for measuring an angle between a line through the two anatomical landmarks and a reference line which is independent of the body; and
the apparatus is connected to an angle indicator.

In the above-mentioned apparatus, said sensor may be constituted by one of an inclinometer and an accelerometer. An inclinometer is an example of a mechanical sensor. An accelerometer may output an electronic/analogue signal or a digital signal.

Said signal transmitter may be constituted by a light-emitting device. The light emitted may be of a common type, for example white light. This is gathered into a narrow light cone of a fixed direction relative to the apparatus. Alternatively, the light may be a laser light of a kind known per se. This has the advantage of the apparatus having low weight. The light cone or the laser light will provide a mark on a surface and this surface may have graduations, for example in the form of lines. Movement of the core of the body may thereby be read as movement of the light mark on the reading surface.

Said sensor may also be constituted by at least a first sensor and a second sensor. This has the effect of more characteristics of a possible movement in the core being measurable. Thus, it can be measured whether the movement is just in one plane, for example the frontal plane, or whether there are movements also in planes perpendicular to the frontal plane. Examples of such movements may be tremblings. Further, the apparatus may be used in exercises in which it is possible that the body may move freely in several planes at the same time. This may be relevant in several exercises with a sling, in which the sling forms an unstable point of support as shown in the examples and figures that follow.

Said second sensor may be constituted by one accelerometer or several accelerometers.

The apparatus as described above may be connected to said angle indicator and a second indicator. Said angle indicator and second indicator may be mechanical indicators, electronic indicators or digital indicators. This means that the indicator may be fixedly mounted on the apparatus or be arranged separate from the apparatus and placed in some other place where it is practical for the test person or a person overseeing the exercise to read it. The at least one sensor of the apparatus may be connected, by means of means known per se, to one or more indicators such as electrical wires, by means of radio signals, by means of high-frequency audiosignals or by means of light signals, in particular infrared light signals. The indicator may output an audiovisual signal of a kind known per se, such as an air bubble in a vial, a pointer arrangement, a screen, a display, a text display, a row of diodes or a sound signal. In one embodiment in which the apparatus is provided with a signal transmitter in the form of a light or a laser light, the indicator is constituted by a surface in the form of a screen or a wall onto which the light core or the laser light is projected. In one embodiment, one indicator indicates the angle or angular deflection through the exercise, whereas another indicator indicates instability caused by muscle trembles. In another embodiment, the indicator alternates between indicating the angle/angular deflection and the instability, either at preset time intervals or by manual switching between one indication or the other. By indicating the angle is meant, here, the absolute angle relative to a predetermined 0° angle, the vertical line forming the 0° angle, for example, and that in such a way that a horizontal line is read as a 90° angle. By indicating the angular deflection is meant, here, that the fixed line, defined as the line through two selected anatomical landmarks, before the exercise is started is set to 0°, and that movement as the exercise is carried out is read relative to this line. The fixed line at the start of the exercise may deviate from the horizontal line or the vertical line.

In the above-mentioned apparatus said sensor and said indicator may be constituted by a bent vial. This has the effect of the sensor and indicator being one and the same device. A bent vial has the advantage of being simple and robust and being usable without any other means, such as an electric power source. The curvature of the vial is of importance for its sensitivity. For example, a vial of the type found in a carpenter's level will not be usable, as small angular deflections will easily cause the bubble to be at the end of the vial, whereby reading is made impossible. The person skilled in the art will know how a bent vial may be formed, with respect to, for example, vial curvature, diameter of the vial tube, bubble size and vial medium, to be appropriately formed in relation to the object of the invention.

The above-mentioned apparatus may be provided with at least one movable adjusting element which is arranged to bring the apparatus into contact with at least one of said two anatomical landmarks. This has the effect of the apparatus being usable independently of the test person's anatomy and size, and the apparatus can be utilized using several anatomical landmarks. The apparatus is fixed to the body by means of, for example, a non-elastic or elastic belt or a strap. To be able to measure movements in a reliable and reproducible way, the apparatus must lie approximately fixed relative to the two anatomical landmarks selected. This is achieved by, for example, there being physical contact between the landmarks and the apparatus. This may be achieved by a stationary part of the apparatus being in contact with one landmark, whereas a movable adjusting element is moved, for example medially or laterally, until the adjusting element is in physical contact with the other landmark. An example of such landmarks is the SIAS points. The pelvic width of children, men and women may vary very much. The adjusting element may be formed as an arm or a bail, for example, and may be movably or displaceably attached to a portion of the apparatus in a manner known per se. The adjusting element may further be provided with a locking device to hold the setting fixed during the performance of the exercise or the measuring. At an end piece which is in contact with the anatomical body point, the adjusting element may further be provided with means that hold the adjusting element fixed. The end piece may be adapted for the anatomical body point and this end piece may be replaceable. In another embodiment, the end piece may have a rough surface which prevents the end piece from slipping. In another design, the end piece may be provided with a hook-and-loop fastener of the loop or hook type catching into a hook-and-loop fastener of the opposite type placed on the anatomical landmark. The latter hook-and-loop fastener may be held in place by a non-elastic or elastic belt spanning the selected anatomical landmarks. Alternatively the whole belt may be formed as such a hook-and-loop fastener. The apparatus may further be provided with a second movable adjusting element. This may be advantageous as, in addition to having a varying distance between the SIAS points for example, test persons are also different when it comes to slimness. It is an advantage that only the anatomical landmarks affect the movements of the apparatus. An additional advantage with two movable adjusting elements is that the portion of the apparatus containing a sensor or sensors, and possibly also an indicator or indicators, can be placed symmetrically relative to the selected anatomical landmarks. The second movable adjusting element may be formed like the first movable adjusting element, and the second element may be attached to a portion of the apparatus in a corresponding way. The apparatus may further be provided with a placement reference element to facilitate and ensure correct placement of the apparatus relative to the anatomical landmarks.

In the above-mentioned apparatus said reference line may be defined by the local gravity. The local gravity will define a horizontal plane perpendicular to the gravitational direction. Further, vertical planes are defined as planes perpendicular to the horizontal plane. This has the advantage of mechanical sensors, such as a bent vial, providing precise measurements even if the bent vial itself does not lie in a vertical plane. It is also possible to provide the apparatus with two or three bent vials which are mutually perpendicular, for readings to be performed regardless of the position of the apparatus. The case of accelerometers is a corresponding one. This is in contrast to, for example, the use of a compass as an angle reader. The accuracy of the compass is dependent on the compass needle pivoting freely, which is achieved when the compass housing is resting on a horizontal surface. As it is desirable to measure movements in all planes and not just in the horizontal plane, the use of a compass as a sensor is not suitable in relation to the object of the invention.

In a second aspect, the invention relates to a method of providing a measured value when measuring the muscular power of a muscle area in a body, by the use of the apparatus described above, the method comprising the steps of:
attaching the apparatus to the body; and
reading at least one first indicator indicating an angle between a line through two anatomical landmarks and a reference line which is independent of the body.

According to the method, the angle reading may be compared with at least one reference value provided. The method provides objective and reproducible values for the muscular power of the core in the form of the capacity that the test person has to control the position of the core when portions of the core are not supported and thereby are passively brought or can actively be brought to rotate. This capacity is expressed as a measured angle, possibly supplemented with measurements of instability and trembles. The same person may be tested repeatedly over time and the values may be compared. Further, values or data from several persons can be collected, forming a basis for a reference material with which values or data from new persons can be compared.

The invention further relates to a method in which the angle reading is recorded manually or electronically in an analogue or digital format. The data recorded may be stored on a manual medium, for example on paper, or on an electronic medium in an analogue or digital format. This has the advantage of enabling the angle readings, results and data to be processed statistically and reproduced in the form of, for example, tables and graphic representations independently of the apparatus and the indicator connected to it. According to the relevant method, the data or signals from the sensor or sensors of the apparatus can be stored electronically or digitally without the data having been read in advance.

In a further embodiment of the method, a second indicator is read, indicating acceleration relative to a reference point. This has the effect of enabling registration of movements other than, for example, the voluntary movement. Muscle tremblings may be registered, and they may spring from muscles or muscle groups that work in the same direction as the voluntary movement or in other directions. Such data may provide additional information on the core power.

Further, the acceleration reading from another indicator may be recorded manually or electronically in an analogue or digital format. The data recorded may be stored on a manual medium, for example on paper, or on an electronic medium in an analogue or digital format.

In a third aspect, the invention relates to the use of the apparatus described above for measuring and documenting the muscular strength of a body core, or for giving a test person feedback on whether a muscle-training exercise is carried out correctly.

The apparatus can be connected to a sound-emitting indicator which outputs an audiosignal when the measured angle gets outside or inside a predefined area, which may mean that a body part is not held in the right position as an exercise is being carried out. This has the advantage of the auditory signal helping the test person to train correctly with the right musculature involved, and with the spine within the neutral zone (lordosis). It may be a conscious or unconscious action for a test person to seek passive support when the test person does not possess sufficient muscular power. By “twisting”/“going out of” the neutral zone/“letting go of lumbar setting”, it is possible to hang on passive structures or recruit larger outer muscles which help the test person to “cheat”. During heavier exercises it may lead to unfortunate and possibly harmful strain if exercises are carried out in which the test person “goes out of” the neutral zone and hangs on passive structures. An important part of a training scheme will be to hold a neutral position under gradually increasing strain. The apparatus may help the test person to train in correct positions, so that the balance between global musculature and deep local musculature is maintained, by checking that the outer global musculature is never allowed to generate more power than for deep local musculature to manage to stabilize the spine under the strain from the global musculature.

The invention is not restricted to the use on humans as the apparatus and method can also be used when examining and training animals.

In one embodiment the invention relates to an apparatus formed as a modified level with an air or gas bubble in an arcuate tube filled with liquid for measuring over a wide range of angles, for example 0°±90°. By putting marks on the tube, it is possible to determine the angular positions and make zones of points and/or passed versus failed zones.

In those cases in which the test participant had vibrations in the body part, this was visually observable, but that will not be very practical in practice, without any further instrumentation objectively picking it up.

In an electronic, possibly digitized embodiment of the apparatus it will also be possible to include, in addition to tilt indication, the indication of such quivering or trembling, in order to provide useful additional information for the therapist/test person. Such indication may appropriately be provided by the use of an accelerometer. Such measuring provides information about how good control of his/her body's core musculature does the test person have. This use of an accelerometer which is thereby measuring “muscular unsteadiness” therefore provides useful additional information. It will be used as a measurement of the control of the activated musculature.

An electronic version of the apparatus will carry the possibility of not only analogue and/or digital indication of tilt, but also indication of said quivering or trembling in percentage values, for example, or absolute values from a “zero-value level”. In additional to analogue and/or digital indication, it will be possible to indicate measurements in the form of colour columns and/or in the form of an acoustic indication. The acoustic indication may be in the form of, for example, increasing whistling sounds, or a voice stating the degree of deviation. An electronic apparatus according to the invention could have connectable data-logging equipment, such as a memory chip, or enable transmission of such information via cable or in a wireless manner to a recording system. Thereby it becomes possible to record a physical condition development over time, either in the form of a material in numbers only and/or in a graphic form.

The apparatus is particularly suitable for indicating obliquities by a fall in the pelvis position. In a “supine abduction test”, for example, the apparatus is attached to a belt around the pelvis of the test person who is then lying on his/her back with just one leg in an unstable point of support (rope from the ceiling). The test works as an element in revealing weak musculature in the abdominal, back and pelvic regions. Participants who are strong will manage to keep the pelvis level) (0°, whereas participants with weak musculature will fall. By means of the apparatus, it is possible in a simpler and more reliable manner to precisely measure the core power of the body. The test person's achievement will be related to, inter alia, any deviation in degrees from the starting point 0°, and the extent of instability (unsteadiness) during the performance of the exercise.

The apparatus is suitable not only for the therapist to perform test measurements at regular intervals, but for the person concerned also to use the apparatus him-/herself for self-checking and motivation during home training of certain agreed exercises. The apparatus may therefore be a useful aid in such solo training, in which the user may get visual and/or auditory feedback on exercises and whether exercises are done correctly.

Doctors, physiotherapists or other specialists could use the apparatus for mapping a person's abdominal and back musculature, such as establishing weak musculature and poor body control, and measuring any change in the power, that is to say improvement and decay. Thus, the apparatus is a useful means for making arrangements for possible physical treatment and/or a necessary training scheme. It may be worth mentioning that as core power seems to be closely linked to back problems, it may increase efficiency to map the patient's muscular power in order then to be able to possibly rule out this potential causal source of the symptoms. If it is found that the patient possesses good power and control of his/her core musculature, it is obvious to look further for mechanical causal sources of the symptoms, for example a prolapse, slipped disc and the like. The method will be very useful in that the core is tested in a manner true to nature and a picture of the patient's functional power capacity is formed. This is in contrast to the existing methods previously referred to.

The apparatus may also be used to register and measure motion patterns during different activities. Studies have shown that an altered recruitment pattern, leading, in turn, to an altered motion pattern, may be closely linked to pathology and pain influence. By logging these movements, it is possible objectively to observe a person's path of motion. For example, the standing, sitting and walking pelvic positions may be logged and useful information be obtained about the test person's recruitment and motion patterns in the pelvic region. For example, a person's change in motion pattern may be established, which, in turn, may provide useful information about a test person's physical health. It is then possible to do testing before and after an injury, for example, or after a training intervention and see whether this pattern has changed.

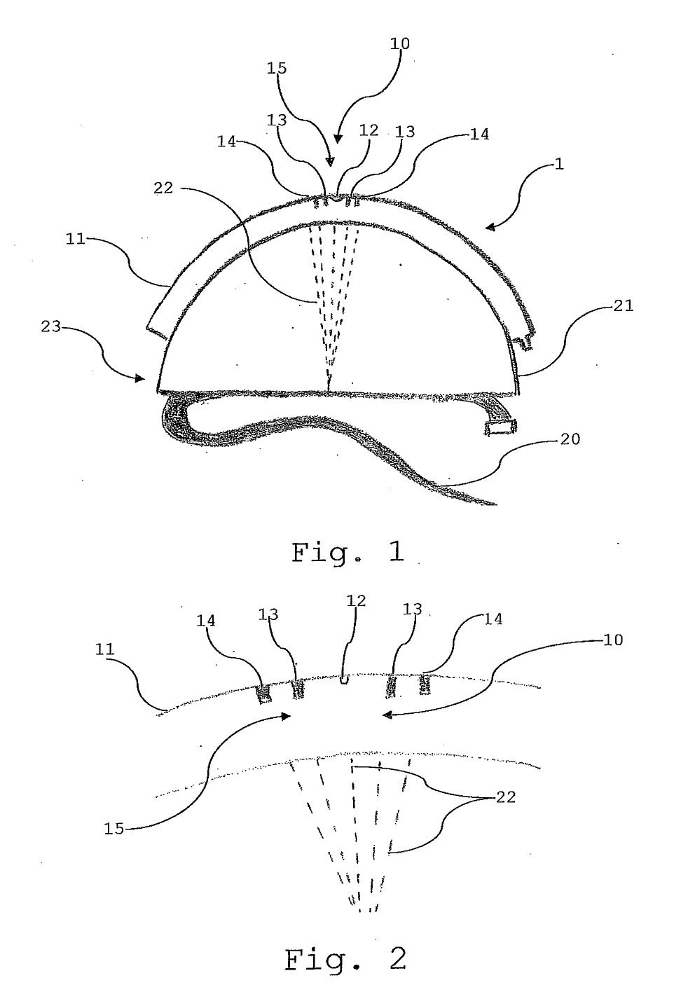

Regardless of whether the apparatus is made in a “mechanical” form with a vial-based solution corresponding to that shown in FIGS. 1 and 2 or by the use of another type of analogue tilt indicator, such as a kind of pendulum pointer, or whether it is made in an electronic form with known types of mechanical/electric, resistive/electric, opto/electric, magneto/electric or capacitive/electric inclinometers, it is basically the use and the design of the inclinometer device that are important aspects of the invention.

In relation to using costly and, in practice, impractical 3D equipment for measuring the tilt of body parts, the apparatus according to the present invention represents a reasonable, reliable and technically simple solution.

To be able to use the apparatus for measurements in connection with different body parts, it may be appropriate to make it bipartite, that is to say a first part, an attachment part, for placement on and attachment to the body, and a second part, a measuring part, consisting of the apparatus itself, containing the inclinometer part and possibly an accelerometer and other equipment, and this in such a way that the two parts can easily be attached to each other. The first part may possibly be available in several variants adapted for the body parts to which it is to be fixed.

It is also important that the first part may be adjustable to different sizes of the same body part. This can be achieved by the attachment part being provided with adjusting elements consisting of one or more of length adjustment elements, width adjustment elements, and level adjustment elements. The level adjustment elements may be arranged at the end regions of the attachment part. The length adjustment elements may be adjustable in thickness in their end regions.

Further, it is important that such an apparatus is light and does not have too high a centre of gravity, so that it feels comfortable wearing it on the body. The apparatus should also be easy to attach to the body and easy to remove after use. The use of lightweight materials may therefore be essential.

It is possible to let both the inclinometer, accelerometer, indicators and threshold circuits, and the associated processor be in the measuring unit which is attached to the attachment part, but it is also conceivable that only the inclinometer and/or accelerometer are/is incorporated in the measuring part, so that the indicators, threshold circuits and the processor are in a separate apparatus housing which may form a connection to the inclinometer and accelerometer in the measuring unit. The apparatus housing may thereby be arranged in such a way, for example, that it will be more convenient for a therapist or the test person him-/herself to watch and also operate it.

Especially for use at the pelvic portion, it is important to have an ergonomically adapted design, so that even with different body shapes, the apparatus will be similarly placed relative to the same anatomical landmarks in different persons. One embodiment of the first part suggests how this can be realized. In another embodiment the bottom portion of the attachment part is formed concave. In a further embodiment, the attachment part may further be provided with a placement reference element.

The use of a logging function could be useful when using the apparatus, not just at a therapist's, for example at a physical institute or at a hospital, but also for home use. For home use, possibly also for professional use, it is also imaginable that the apparatus may be remote-controllable for activation and deactivation.

In what follows is described an example of a preferred embodiment which is visualized in the accompanying drawings, in which:

FIG. 1 shows a bent vial meant for attachment to the body;

FIG. 2 shows, on an enlarged scale, a detail of FIG. 1;

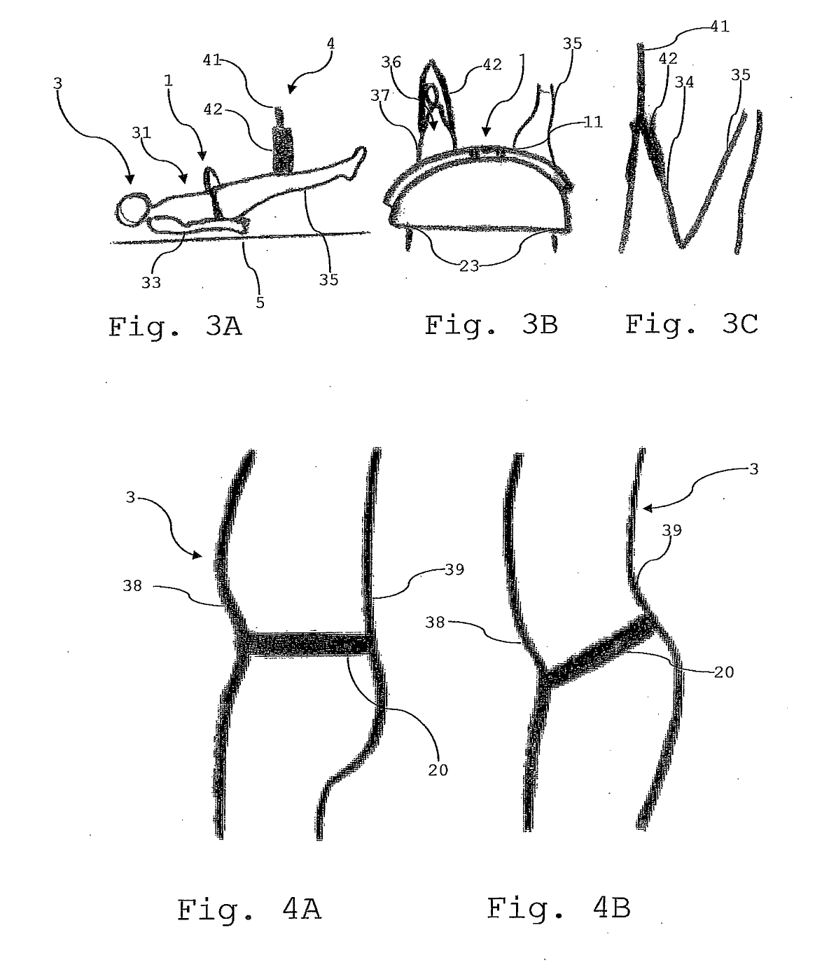

FIGS. 3a-c show the performance of a “supine abduction test”;

FIGS. 4a, b show the measuring of pelvic rotation during the straightening of lordosis;

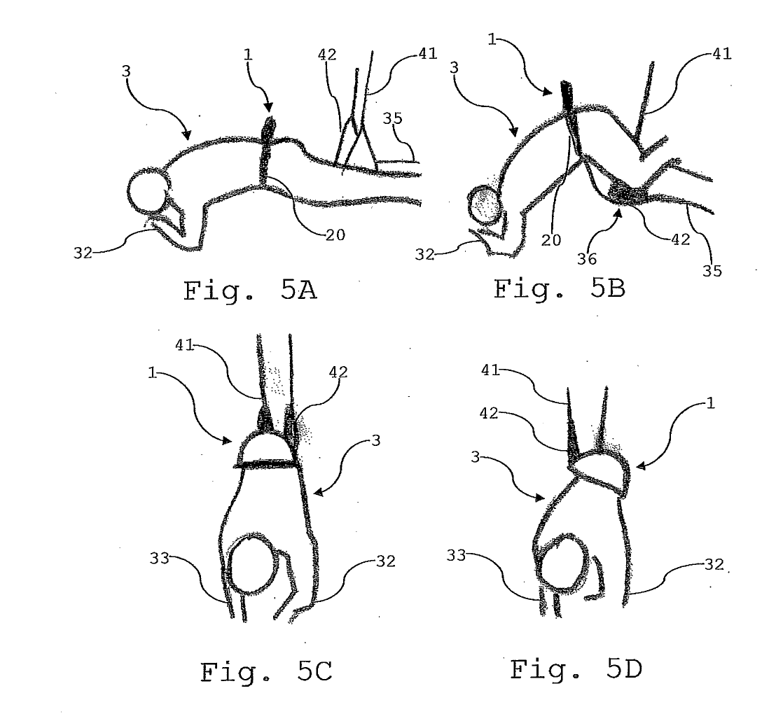

FIGS. 5a-d show the performance of an “abdominal bridge” test;

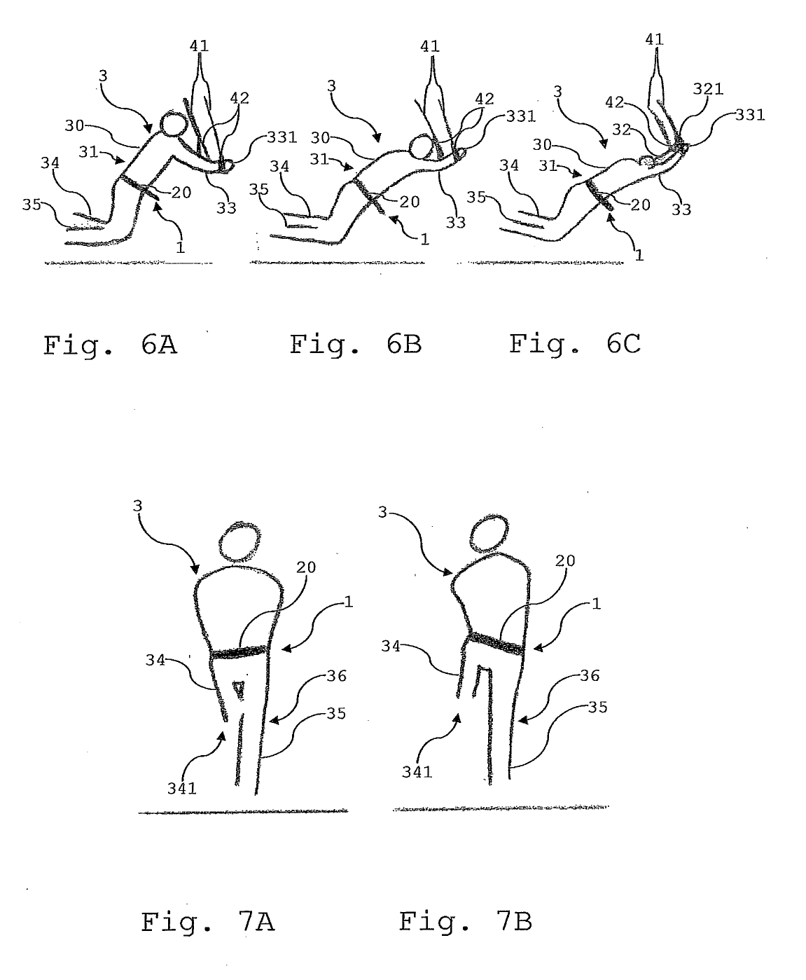

FIGS. 6a-c show forward-leaning “weak-link” test;

FIGS. 7a, b show the measuring of maximum pelvic rotation in the frontal plane, the Tredelenburg test;

FIGS. 8a-c show the measuring of maximum pelvic rotation in a transverse plane;

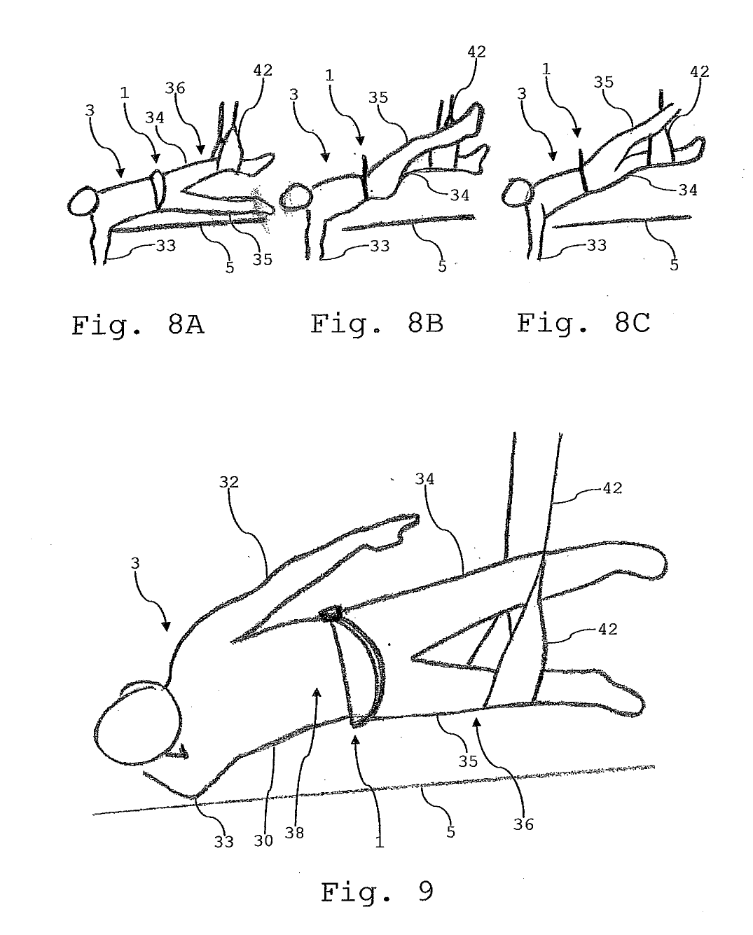

FIG. 9 shows a lateral bridge test with abduction;

FIGS. 10a, b show press-ups (push-ups) in a sling; and

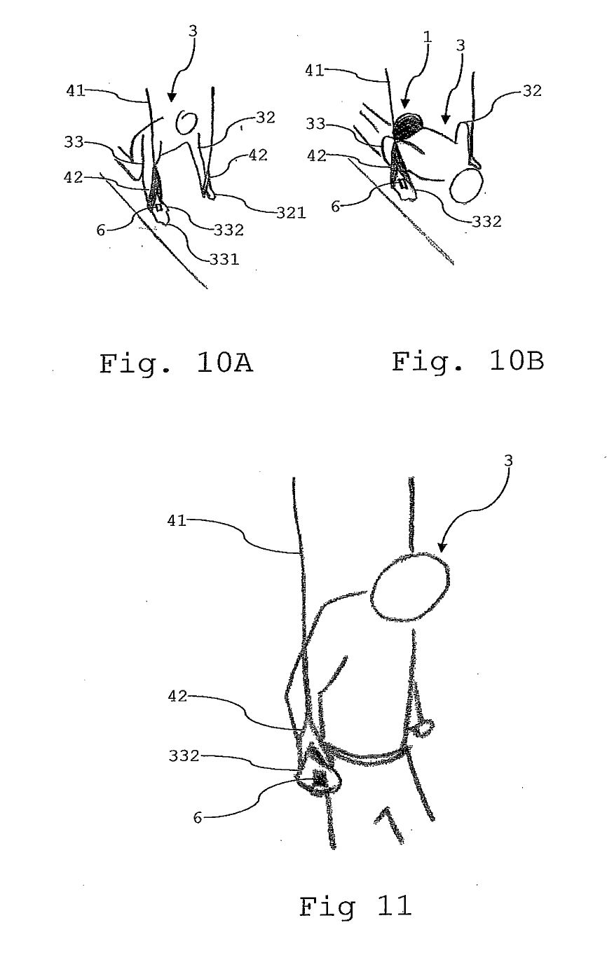

FIG. 11 shows arm press in a sling.

According to the invention, an apparatus as initially mentioned is provided, which is characterized by a measuring part including an inclinometer with at least one tilt indicator, and an attachment part adapted for attachment to a body part or a body area related to the muscle area to be measured.

### EXAMPLE 1

An apparatus 1 in accordance with the invention may consist of a 3 mm aluminium plate 21, a strapping belt 20 and a 16 mm closed tube 11 filled with 96% spirit, the closed tube 11 being both a sensor 10 for tilt and an indicator 15 for reading tilt. The aluminium plate 21 is formed as a semicircle with a radius of 15 cm and is angled at 90° at the bottom (not shown), so that a narrow rectangle appears, which can be used as a base (not shown). The base works as an attachment surface (not shown) for the belt 20 by the use of rivets, for example. In an alternative embodiment, the belt 20 may be fixed to the base with glue. The belt 20 is used to strap the apparatus 1 to a test person's 3 body at the pelvis. In this example, the belt 20 and base form the attachment part 23 of the apparatus 1. The aluminium plate 21 works as a protractor marked with degree marks 22 with a mark for a vertex, 0°, and with a graduation for every 2.5° up to 30°, both to the right and left of the vertex, as shown in further detail in FIG. 2. The closed tube 11 contains a gas bubble 12 with a diameter of approximately 5 mm. In another embodiment, the bubble 12 has a diameter of 2-3 mm. The closed tube 11 is provided with marks 13 for 2.5° on either side of the vertex of the closed tube 11 when the apparatus 1 is resting on a horizontal surface. Correspondingly, the closed tube 11 is provided with marks 14 indicating 5°. The closed tube 11 is thus a level or an inclinometer.

In an alternative embodiment said closed tube 11 filled with liquid may be replaced by, for example, a pendulum-shaped pointer (not shown). The pendulum-shaped pointer may further be placed in a chamber filled with liquid (not shown).

In an alternative embodiment it is desirable ergonomically to adapt the apparatus 1 in such a way that it may be used on different-size persons 3, and in such a way that it will be possible to adjust the position of the apparatus 1 to the desired anatomical landmarks in order to achieve more accurate measurements. This may be achieved by the apparatus 1 being provided with one or more movable adjusting elements (not shown) which are displaceable or movable relative to the aluminium plate 21. The adjusting element may be tubular and the adjusting element is movable in a groove or a sleeve attached to the aluminium plate 21 or another suitable part of the apparatus 1. The adjusting element may be fixed in the desired position with an attachment screw, for example. Alternatively, the adjusting element may be rotatably attached to the apparatus 1 and provided with a resilient device holding the adjusting element clamped against the anatomical landmark. Other, alternative solutions exist and they will be known to the person skilled in the art.

### EXAMPLE 2

The “supine abduction test” is an exercise which may appropriately be performed on a training apparatus 4, for example of the type known under the trade name “Redcord Trainer™”. This is an apparatus consisting of two adjustable ropes 41 hanging from a ceiling or other point of attachment (not shown). Each rope 41 has a sling 42 at its lower end. The test challenges the core 31 in three planes of motion. The test is unstable; that is to say, the core 31 is not supported by any surface. The test uses the test person's 3 own weight, body weight, as a counterweight, which has advantages previously described. The exercise is well known in physiotherapist and rehabilitation circles as both a training exercise and a clinical test. The test is relatively new in a scientific connection. Comprehensive reliability results are not yet available. When the test is performed in the, for the time being, usual way, there is no measuring method or measuring instrument which can objectively provide information about the condition of the core or the core power.

It is therefore desirable to develop this test further into being more objective and easier to assess, by providing a measuring method and/or a measuring instrument which may solve this objectivity problem.

The “supine abduction test” was standardized in such a way that in the starting position, the test person 3 was lying on his/her back with one leg 34 in the sling 42. The other leg 35 was resting on a bench 5 (not shown in this position). The arms 32 and 33 were resting along the sides, palms facing the bench 5 (FIG. 3a). The sling 42 was 10 cm wide and was placed at the transition between the knee 36 and the thigh 37, so that the centre of the sling 42 was 5 cm proximal with respect to the centre of the patella. The lower end of the sling 42 was 20 cm above the bench 5 in a state influenced by weight.

The two “spinae iliacae anteriores superiores” were selected as anatomical landmarks.

The apparatus 1 was placed halfway between the two SIAS points (FIG. 3b); that is to say, the end points of the attachment part 23 of the apparatus 1 were resting on the opposite edges of the pelvis. It was important that the apparatus 1 was lying fixed relative to the two SIAS points as it was the line between these two reference points relative to a horizontal line that was desirably to be measured.

In an alternative embodiment, the attachment part 23 of the apparatus 1 is made concave on the underside, so that, when strapping it on, the test person's 3 abdominal portion does not create any problems.

The test person 1 extended his/her leg 34 which was supported by the sling 42. The free test leg 35 was lifted up beside and against the “slinged leg” 34 (FIG. 3a). The pelvis was raised so that the test person's 3 body was in a plank position, (FIG. 3a) resting on its shoulder portion and in the sling 42. By means of the inclinometer 11, the test person 3 placed him-/herself in a leveled position (FIG. 3b), so that the line between the two SIAS points was horizontal. The test person 3 steadily and controlledly abducted his/her test leg 35 out into a thirty-degree angle (FIG. 3c) while the test person 3 still tried to hold the pelvis level.

The test person 3 was assessed on a scale of points from one to ten, one being “Very bad” and ten being “Very good”. Five and lower were assessed as “Failed”, six and higher as “Passed”. The assessment was done at the time when the leg 35 was in a thirty-degree abducted position. To achieve six or higher, the vial bubble 12 had to be within the 2.5 degrees' marks 13 (FIG. 2). Additional points were allocated for the stability of the bubble 12. If the bubble 12 was approximately stationary, centred between the marks 13, the test person 3 was allocated the highest score, ten. If the bubble 12 was very unstable, the test person 3 was allocated the score of six. In those cases in which the bubble 12 remained outside the marks 13, the participant was allocated a score of five or lower. If the bubble 12 was just outside the marks 13, the participant was allocated a score of five. In those cases in which the bubble 12 was very unstable and slid way outside the marks 13 and 14, the lowest total number of points was allocated. The whole scale of points was used with these directory scoring criteria.

The test person 3 was first taken through the path of motion and given instruction while the test supervisor was physically holding the pelvis (SIAS points) in the correct position. This was because the test person 3 should experience the correct working position. Then the test person raised him-/herself into position and adjusted him-/herself into the correct starting position by means of visual feedback from the inclinometer 11. Six test trials were carried out, two by two being carried out without a break. In repetitions one, two and five, the test person 3 was allowed to inform him-/herself on the position of the pelvis by watching the bubble 12 of the inclinometer 11 during the entire test. In repetitions three, four and six, the test person was to look up at the ceiling after the test person 3 had reached the levelled starting position. The test person 3 then had to utilize proprioceptive feedback instead of visual feedback while trying to hold the pelvis in the same position. Between each repetition the test person 3 positioned him-/herself all over again. The left side was tested first, then the right side with another six test trials.

The tests performed showed that a good and objective reference value was obtained. The repeated measurements were comparable and reproducible. In addition, it was quickly and unambiguously discovered if the test person 3 was too weak to carry out the test or prevented from carrying out the test because of pain, for example pain in the lower back.

Thus, the invention is suitable for measuring that a body part or a body area is in a fixed reference position when test measurements are carried out. It is also possible to take the same reference position in subsequent tests and compare the measuring results obtained according to the invention. Further, it is possible by means of the invention to demonstrate acceptable deviations from the default value, including deviations due to quivering in the body. Further, the invention is also usable for determining the inclination of a body part during the performance of a dynamic test as well as a static test, in order thereby to create a reference value with respect to subsequent measurements.

### EXAMPLE 3

The example shows the straightening of lordosis (FIGS. 4a and b). When this sway is straightened in a body-weight position, the sway, in other respects, being a natural one, deep core musculature, that is to say a local stabilizer system, is activated and the body is trained in a position in which, normally, the spine is within the neutral zone. Cases in which there is deviation may be if the test person straightens so much that he or she gets tension in passive structures, thereby going into the elastic zone. The apparatus 1 is very useful in connection with training as the test person 3 may get objective feedback on what is the position of the pelvis. The apparatus 1 can further be used to measure mobility deflections in a first test and subsequently in repeated testing. The apparatus 1 is a pedagogic aid for the therapist to visualize the exercise and get the test person him-/herself to understand the movement. Effective training of near-joint and local musculature is dependent on the test person 3 keeping his/her spine in the neutral zone when performing the exercises. Such exercises may be sling training, “core bar” training, training with a bosu ball or a physioball, and a number of other exercises. An apparatus 1 according to the invention is attached to the test person's 3 body and is fixed relative to the anatomical landmarks navel 38 and 5^{th} lumbar vertebra 39. In the starting position, that is to say when the back is in the neutral zone (FIG. 4a), the apparatus 1 may be zeroed, so that movement of the spine out of the neutral zone registers as a deviation from the “0” position (FIG. 4b). In this example, the apparatus 1 measures the angle between the line through the navel 38 and the 5th lumbar vertebra 39 and a line in the horizontal plane or a line in a vertical plane.

Some people/patients have increased lordosis, that is to say more sway in the lower part of their backs than what is normal. This posture may be a result of bad habits, weak musculature or a result of an injury. The posture may be influenced or normalized by training of, inter alia, the local stabilizer system including the psoas. For these patients, training with the apparatus may be an important part of their treatment.

### EXAMPLE 4

The example shows an “abdominal bridge exercise”. The test person 3 is resting on his/her arms 32, 33 and with one knee 36 in a sling 42 (FIG. 5a) in an approximately horizontal position. The exercise is performed by the test person 3 bending and moving both legs 34, 35 in under his/her body (FIG. 5b) while attempting to hold the pelvis in a horizontal position (FIG. 5c) at the same time. By too poor core stability, the pelvis will move in the transverse plane around a longitudinal axis (FIG. 5d). The apparatus 1 is fixedly attached relative to the SIAS points across the person's back, as shown in FIG. 5. The core stability assessment may be carried out with the same allocation of points as in example 2.

### EXAMPLE 5

The example shows the performing of a “weak-link” test. The test person 3 is kneeling with his/her legs 34, 35 together, holding a sling 42 in either hand 321, 331. The arms 32, 33 are straight (FIG. 6a). The test person 3 steadily and controlledly moves his/her upper body 30 forwards while the arms 32, 33 are still straight (FIGS. 6 b and c). If there are “weak links” in the core 31, the pelvis will rotate to one of the sides in the transverse plane around the longitudinal axis, or the test person attempts to compensate by rotating around the frontal axis, taking a “banana posture”. It is then possible to establish that there are weaknesses or possible lateral differences. This is a sign of some musculature not making a sufficient contribution. It may also have other causes, such as fear of pain. The apparatus 1 may be attached across the test person's abdominal portion, but it may also be appropriate to attach the apparatus 1 across the small of the test person's 3 back with the SIAS points as anatomical landmarks. The apparatus 1 may be used as in example 2 for giving points. It may also be used for measuring the angle of rotation, and repeated measurements over time may quantify training progress.

### EXAMPLE 6

The example shows the measuring of maximum pelvic rotation in the frontal plane around the sagittal axis, the so-called Tredelenburg test. The test person 3 is standing on one leg 35 while the other leg 34 is lifted with the blade 341 of the foot to the height of and against the knee 36 of the straight leg 35 (FIG. 7a). The test person 3 voluntarily rotates the pelvis in the frontal plane around the sagittal axis as high up on the free side as possible (FIG. 7b). The apparatus 1 is attached across the small of the back with the SIAS points as anatomical landmarks (FIGS. 7a, b). Maximum pelvic rotation is measured as the angle between the SIAS line and the horizontal plane. The exercise is repeated on the other side. The apparatus 1 quantifies the maximum pelvic rotation and this can be used to measure training progress and also be compared with a norm material or reference material.

### EXAMPLE 7

The example shows the measuring of voluntary pelvic rotation in the transverse plane around the longitudinal axis. The test person 3 is supine with his/her arms 32, 33 straight out to the sides. One leg 34 is supported by a sling 42. The sling 42 is positioned distally of the knee 36. The lower end of the sling 42 is about 30 cm above the surface 5. The free leg 35 is extended and is resting on the surface 5 (FIG. 8a). The test person 3 performs the exercise by rotating his/her pelvis in the transverse plane while the free leg 35 is kept straight. During the rotation, the free leg 35 is first raised to the height of and parallel to the leg 34 in the sling 42. By further rotation, the free leg 35 gets above the leg 34 in the sling 42 (FIGS. 8b and c). The apparatus 1 is attached across the abdomen with the SIAS points as anatomical landmarks (FIG. 8). Maximum pelvic rotation is measured as the angle between the SIAS line and the horizontal plane. The exercise is repeated on the other side. The apparatus 1 quantifies the maximum pelvic rotation and this can be used to measure training progress and also be compared with a norm material or reference material. The exercise may further be used to measure muscular power by measuring the time from the lowermost to the uppermost position and the number of repetitions the test person 3 is able to carry out within a correctly performed exercise.

### EXAMPLE 8

The example shows a lateral bridge test with abduction. The test person 3 is in a lateral position, resting on his/her bottom arm 33. The bottom leg 35 is supported by the sling 42. The sling is positioned distally of the knee 36. The lower end of the sling 42 is about 10 cm above the surface 5. The free leg 34 is straight, resting on the bottom leg 35. The exercise is carried out by moving the top leg 34 outwards in the frontal plane; that is to say, upwards in an abducting movement. The top arm 32 may also be moved outwards in the frontal plane in an abducting movement (FIG. 9). The apparatus 1 is attached across the abdomen with the SIAS points and the navel 38 and the 5^{th} lumbar vertebra (not shown) as anatomical landmarks.

In this exercise the pelvis may rotate in three planes. Firstly, the pelvis may rotate in the frontal plane. That is to say, the test person 3 tips over to one of the sides around the longitudinal axis. This will be measured by the apparatus as a change in the angle between the line through the SIAS points and the vertical plane approximately equal to the frontal plane. Alternatively, this may be measured as a change in the angle between the navel 38 and the 5^{th} lumbar vertebra (not shown) and the horizontal plane.

Further, the pelvis may rotate around the sagittal axis in the frontal plane; that is to say, the lower hip crest sags towards the surface 5. This will be measured by the apparatus 1 as a change in the angle between the line through the SIAS points and the horizontal plane.

The pelvis may further rotate in such a way that the upper body 30 and legs 34, 35 do not form a straight extension; that is to say, there will be a bend at the hip. This will be measured by the apparatus 1 as a rotation around the frontal axis or as a change in the angle between the line through the navel 38 and the 5^{th} lumbar vertebra (not shown) and the vertical plane approximating the transverse plane.

The pelvis may further rotate by the vertebral column coming out of the neutral position, that is to say increased lordosis. This can be measured as a rotation around the frontal axis or as a change in the angle between the line through the navel 38 and the 5^{th} lumbar vertebra (not shown) and the vertical plane approximating the transverse plane.

So, to make satisfactory measurements in this exercise, more than one sensor is needed. The apparatus 1 may thus include three accelerometers, measuring in three perpendicular planes or around three rotational axes with individual reading and possible recording.

### EXAMPLE 9

The example shows press-ups (pus-ups) in the sling 42 (FIGS. 10a, b). This is a “weak-link” test. The apparatus 1 is attached across the small of the back and measures the angle between the line through the SIAS points and the horizontal plane. The test shows whether the arms 32, 33 do an equal amount of work and whether the core musculature contributes equally on both sides. In addition to sensors in the apparatus 1, further sensors may additionally be attached to the test person's 3 body. For example, a sensor 6 in the form of one or more accelerometers may be attached to the back(s) 332 of one or both hands 321, 331 of the test person 3 (FIGS. 10a and b). It or they will work as an unsteadiness indicator and provide data on how much control the test person 3 has during the exercise. If the test person 3 is trembling much, this is a sign of poor coordination and stabilization capacities.

### EXAMPLE 10

The example shows arm press in a sling (FIG. 11). The apparatus may be attached across the chest (not shown), so that the anatomical landmarks lie on one of the pairs of ribs. The apparatus 1 measures the angle between the line through the anatomical landmarks and the horizontal plane. As in example 9, one or more sensors 6 may be attached to the back(s) (332) of one or both hands of the test person 3.

1. Apparatus for use when assessing muscular strength and body posture in a body core, wherein
at least a portion of the apparatus is arranged to be attached to a body;
the apparatus is arranged to be substantially fixedly positioned relative to two anatomical landmarks;
the apparatus is provided with at least one of a sensor and a signal transmitter for measuring an angle between a line through the two anatomical landmarks and a reference line which is independent of the body; and
the apparatus is connected to an angle indicator.

2. The apparatus in accordance with claim 1, wherein said sensor or signal transmitter is constituted by one of an inclinometer and an accelerometer.

3. The apparatus in accordance with claim 1, wherein said signal transmitter is constituted by a light-emitting device.

4. The apparatus in accordance with claim 1, wherein said sensor is formed at least by a first sensor and a second sensor.

5. The apparatus in accordance with claim 4, wherein the second sensor is constituted by one accelerometer or several accelerometers.

6. The apparatus in accordance with claim 1, wherein said angle indicator is constituted by at least a first indicator and a second indicator.

7. The apparatus in accordance with claim 1, wherein said sensor and said indicator is constituted by a bent vial.

8. The apparatus in accordance with claim 1, wherein the apparatus is provided with at least one movable adjusting element arranged to bring the apparatus into contact with at least one of said two anatomical landmarks.

9. The apparatus in accordance with claim 1, wherein said reference line is defined by the local gravity.

10. A method of providing a measured value when measuring the muscle power of a muscle area in a body by the use of an apparatus in accordance with claim 1, wherein the method comprises the steps of:
attaching the apparatus to the body; and
reading at least one first indicator indicating an angle between a line through two anatomical landmarks and a reference line which is independent of the body.

11. The method in accordance with claim 10, wherein the angle reading is compared with at least one reference value provided.

12. The method in accordance with claim 10, comprising reading a second indicator indicating acceleration relative to a reference point.

13. Use of an apparatus in accordance with claim 1 to measure the muscular strength of a body core, or to give a test person feedback on whether a muscle-training exercise is being performed correctly.

14. The use of an apparatus in accordance with claim 1 to document the muscular strength of a body core.
